(19) United States
(12) Patent Application Publication
(10) Pub. No.: US 2014/0031579 A1
(43) Pub. Date: Jan. 30, 2014

(54) METHOD OF PRODUCING NORBORNANEDICARBOXYLIC ACID ESTER

(71) Applicants: Hiroyuki Kawakami, Ichihara-shi (JP); Ken-ichi Tominaga, Ushiku-shi (JP); Shigeru Shimada, Tsukuba-shi (JP); Kazuhiko Sato, Tsukuba-shi (JP)

(72) Inventors: Hiroyuki Kawakami, Ichihara-shi (JP); Ken-ichi Tominaga, Ushiku-shi (JP); Shigeru Shimada, Tsukuba-shi (JP); Kazuhiko Sato, Tsukuba-shi (JP)

(73) Assignees: National Institute of Advanced Industrial Science and Technology, Tokyo (JP); HITACHI CHEMICAL COMPANY, LTD.

(21) Appl. No.: 14/110,552

(22) PCT Filed: Apr. 13, 2012
(86) PCT No.: PCT/JP2012/060182

§ 371 Date: Oct. 8, 2013

(30) Foreign Application Priority Data
Apr. 14, 2011 (JP) 2011-090168

(51) Int. Cl. C07C 67/347; C07C 67/48

(52) U.S. Cl. CPC C07C 67/347; C07C 67/48
USPC 560/120

(57) ABSTRACT

A method of producing a norbornanedicarboxylic acid ester, the method including a step of reacting a norbornadiene and a formic acid ester in the presence of a ruthenium compound, a cobalt compound, a halide salt and a basic compound.

### TECHNICAL FIELD

The present invention relates to a method of producing a norbornanedicarboxylic acid ester.

### BACKGROUND ART

Conventionally, aromatic epoxy resins have been widely used as the resins for optical members used in optoelectronic equipment and the like, due to their superior heat resistance and mechanical properties during mounting processes onto electronic substrates or the like or during other high-temperature operations, and also due to their versatility. However, in recent years, even in the field of optoelectronic equipment, the use of high-intensity lasers, blue light and near ultraviolet light has expanded considerably, and resins that exhibit levels of transparency, heat resistance and light resistance superior to those of conventional resins are now being demanded.

Aromatic epoxy resins generally exhibit a high degree of transparency to visible light, but are unable to achieve satisfactory transparency in the ultraviolet to near ultraviolet region. Further, cured products formed from an alicyclic epoxy resin and an acid anhydride exhibit comparatively high transparency in the near ultraviolet region, but suffer other problems such as susceptibility to discoloration upon exposure to heat or light, and therefore improvements in heat resistance and ultraviolet discoloration resistance are required. In light of these circumstances, a variety of epoxy resins are being investigated.

On the other hand, heat-resistant resin such as polyamides and polyesters exhibit not only good heat resistance, but also excellent insulating properties, light resistance and mechanical properties, and they are therefore widely used in the electronics field as surface protective films and interlayer insulating films and the like for semiconductor elements. Among such resins, polymers having an alicyclic structure also exhibit excellent transparency in the ultraviolet region, and are therefore starting to be investigated as materials for optoelectronic equipment and various types of displays. Dicarboxylic acids having a norbornane structure and derivatives thereof are being actively used as the raw material monomers for these polymers.

However, norbornanedicarboxylic acid dimethyl ester, which is a derivative of a dicarboxylic acid having a norbornane structure, is generally obtained by subjecting cyclopentadiene and an acrylic acid ester to a Diels-Alder reaction to obtain a norbornene monocarboxylic acid ester, and then adding a carboxylic acid ester to the unsaturated bond. In this Diels-Alder reaction, an exo/endo mixture having a large endo isomer content is obtained. However, it is known that norbornane derivatives having a polar functional group at the endo position degrade the polymerization activity of catalysts (for example, see Patent Document 1), and therefore an exo/endo mixture having a large exo isomer content is desirable.

An example of a method that has been proposed to address the issues outlined above is a method of producing an exo-norbornene monocarboxylic acid methyl ester by subjecting cyclopentadiene and methyl acrylate to a Diels-Alder reaction under high-temperature conditions of 160 to 300° C. (for example, see Patent Document 2). However, in this production method, a problem arises in that the methyl acrylate polymerizes under the high-temperature conditions.

Further, a method has been proposed for isomerizing an endo-norbornene monocarboxylic acid ester in the presence of a basic catalyst such as a metal alkoxide to obtain the exo isomer (for example, see Patent Document 3), but the resulting exo isomer content is only about 55 mol %, which is still not entirely satisfactory.

### CITATION LIST

### Patent Literature

Patent Document 1: JP 2003-128766
Patent Document 2: WO 03/035598
Patent Document 3: JP 2007-261980

### SUMMARY OF INVENTION

### Technical Problem

An object of the present invention is to provide a method for efficiently producing a norbornanedicarboxylic acid ester having a high exo isomer content.

### Solution to Problem

As a result of intensive research aimed at achieving the aforementioned object, the inventors of the present invention discovered that by reacting norbornadiene and a formic acid ester in the presence of a catalyst system composed of a combination of a ruthenium compound, a cobalt compound, a halide salt and a basic compound, a norbornanedicarboxylic acid ester having a high exo isomer content could be obtained with good efficiency, and they were therefore able to complete the present invention.

The present invention relates to a method of producing a norbornanedicarboxylic acid ester, comprising a step of reacting a norbornadiene and a formic acid ester in the presence of a ruthenium compound, a cobalt compound, a halide salt and a basic compound.

One embodiment of the present invention provides a method of producing a norbornanedicarboxylic acid ester, wherein the norbornanedicarboxylic acid ester is represented by a formula (I) or a formula (II) shown below:

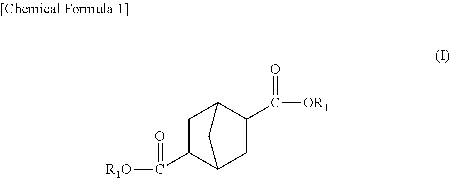

(In the formula, each R₁ independently represents an alkyl group of 1 to 5 carbon atoms, a vinyl group, or a benzyl group.)

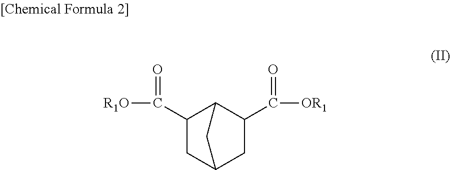

(In the formula, each R₁ independently represents an alkyl group of 1 to 5 carbon atoms, a vinyl group, or a benzyl group.)
and the method comprises a step of reacting norbornadiene represented by a formula (III) shown below:

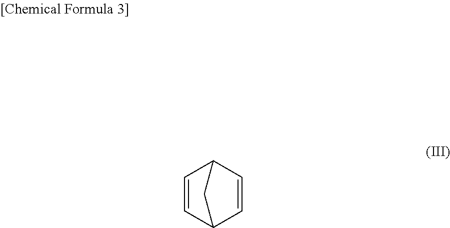

and a formic acid ester represented by a formula (IV) shown below:

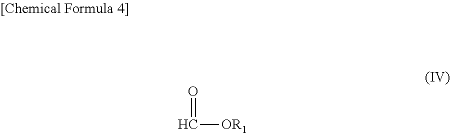

(In the formula, R₁ represents an alkyl group of 1 to 5 carbon atoms, a vinyl group, or a benzyl group.) in the presence of a ruthenium compound, a cobalt compound, a halide salt and a basic compound.

Further in one embodiment of the present invention, a ruthenium complex compound having a carbonyl ligand and a halogen ligand can be used as the ruthenium compound. Further, a quaternary ammonium salt can be used as the halide salt. Moreover, a tertiary amine compound can be used as the basic compound.

In one embodiment of the present invention, when the norbornadiene and the formic acid ester are reacted, a phenol compound and/or an organohalogen compound may also be present in the reaction system.

Moreover, one embodiment of the present invention relates to a method of producing an exo-norbornanedicarboxylic acid ester, comprising a step of separating the norbornanedicarboxylic acid ester obtained using the aforementioned method of producing a norbornanedicarboxylic acid ester into an endo-norbornanedicarboxylic acid ester and an exo-norbornanedicarboxylic acid ester.

The present application is related to the subject matter disclosed in prior Japanese Application 2011-090168 filed on Apr. 14, 2011, the entire content of which is incorporated herein by reference.

### Advantageous Effects of Invention

According to the present invention, a norbornanedicarboxylic acid ester having a high content of the desired exo isomer can be produced efficiently, in a single step reaction, using inexpensive raw materials.

### BRIEF DESCRIPTION OF DRAWINGS

FIG. 1 is a ¹³C-NMR spectrum of an exo-norbornanedicarboxylic acid methyl ester obtained in Example 4.

FIG. 2 is a ¹³C-NMR spectrum of the exo-norbornanedicarboxylic acid methyl ester obtained in Example 4.

FIG. 3 is a ¹H-NMR spectrum of the exo-norbornanedicarboxylic acid methyl ester obtained in Example 4.

FIG. 4 is a ¹H-¹³C HSQC spectrum of the exo-norbornanedicarboxylic acid methyl ester obtained in Example 4.

FIG. 5 is a ¹H-¹H COSY spectrum of the exo-norbornanedicarboxylic acid methyl ester obtained in Example 4.

FIG. 6 is a ¹H-¹³C HMBC spectrum of the exo-norbornanedicarboxylic acid methyl ester obtained in Example 4.

FIG. 7 is a ¹H-¹H NOESY spectrum of the exo-norbornanedicarboxylic acid methyl ester obtained in Example 4.

FIG. 8 is a ¹H-NMR spectrum of an exo-norbornanedicarboxylic acid obtained in Reference Example 1.

### DESCRIPTION OF EMBODIMENTS

The present invention is described below. The present invention provides a method of producing a norbornanedicarboxylic acid ester, the method having a step of reacting a norbornadiene and a formic acid ester in the presence of a ruthenium compound, a cobalt compound, a halide salt and a basic compound.

One embodiment of the present invention provides a method of producing a norbornanedicarboxylic acid ester, wherein the norbornanedicarboxylic acid ester is represented by a formula (I) or a formula (II) shown below:

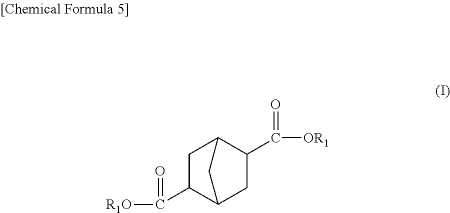

(In the formula, each R₁ independently represents an alkyl group of 1 to 5 carbon atoms, a vinyl group, or a benzyl group.)

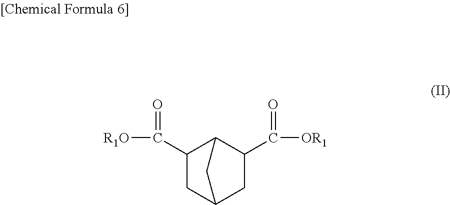

(In the formula, each R₁ independently represents an alkyl group of 1 to 5 carbon atoms, a vinyl group, or a benzyl group.)
and the method having a step of reacting norbornadiene represented by a formula (III) shown below:

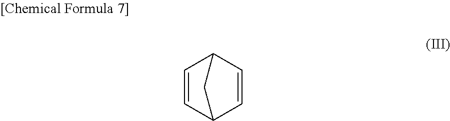

and a formic acid ester represented by a formula (IV) shown below:

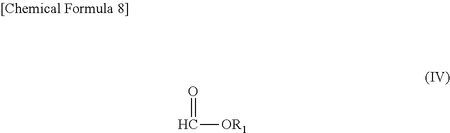

(In the formula, R₁ represents an alkyl group of 1 to 5 carbon atoms, a vinyl group, or a benzyl group.) in the presence of a ruthenium compound, a cobalt compound, a halide salt and a basic compound.

Examples of the alkyl group of 1 to 5 carbon atoms in the formulas (I) and (II) include a methyl group, ethyl group, propyl group, butyl group and pentyl group, and these groups may be either linear or branched. The reaction between norbornadiene represented by the formula (III) and the formic acid ester represented by the formula (IV) yields a norbornanedicarboxylic acid ester containing at least one of a norbornanedicarboxylic acid ester represented by the formula (I) and a norbornanedicarboxylic acid ester represented by the formula (II).

### (Formic Acid Ester)

There are no particular limitations on the types of formic acid esters that can be used as a raw material. For example, the formic acid ester may be selected appropriately from among methyl formate, ethyl formate, propyl formate, isopropyl formate, butyl formate, isobutyl formate, amyl formate, isoamyl formate, vinyl formate, and benzyl formate and the like. From the viewpoints of cost and reactivity, methyl formate is preferable. In the present invention, a single formic acid ester may be used alone, or a combination of a plurality of formic acid esters may be used.

In the present invention, a catalyst system is used that contains 4 essential components, namely a ruthenium compound, a cobalt compound, a halide salt and a basic compound. As is evident from the examples described below, in the present invention, the combination of a ruthenium compound, a cobalt compound, a halide salt and a basic compound enables the desired object to be achieved. Although not bound by theory, it is thought that in the norbornadiene esterification of the present invention, the ruthenium compound cleaves the C—H bond of the formic acid ester, and subsequent reaction proceeds via a reaction with the cobalt compound added to the unsaturated group of norbornadiene, with this reaction being accelerated by the halide salt and the basic compound. A specific description of each of these compounds is provided below.

### (Ruthenium Compound)

There are no particular limitations on the types of ruthenium compounds that can be used in the present invention, provided the compound contains ruthenium. Examples include ruthenium complex compounds having a structure in which ligands are bonded to a ruthenium atom. In one embodiment of the present invention, a ruthenium complex compound having both a carbonyl ligand and a halogen ligand within the molecule is preferable. Examples of the halogen include chlorine, bromine and iodine, and of these, chlorine is preferable. Specific examples of this type of ruthenium complex compound include various types of compounds, including ruthenium carbonyl halogen complexes such as [Ru(CO)₃Cl₂]₂ and [Ru(CO)₂Cl₂]ₙ (wherein n represents an integer of 1 or greater), and ruthenium carbonyl halogen complex salts having an anion such as [Ru(CO)₃Cl₃]⁻, [Ru₃(CO)₁₁Cl]⁻ or [Ru₄(CO)₁₃Cl]⁻ as a counter anion. Salts having an aforementioned counter anion may have a metal ion of an alkali metal or an alkaline earth metal or the like as the counter cation. Specific examples of these alkali metals and alkaline earth metals include lithium, sodium, potassium, rubidium, cesium, calcium and strontium. Among the compounds mentioned above, from the viewpoint of improving the reactivity, ruthenium carbonyl halogen complexes such as [Ru(CO)₃Cl₂]₂ and [Ru(CO)₂Cl₂]ₙ are particularly preferable.

The ruthenium compound can be produced in accordance with methods that are known in the technical field, or can be procured as a commercially available product. Further, [Ru(CO)₂Cl₂]ₙ can be produced using the method disclosed in M. J. Cleare, W. P. Griffith, J. Chem. Soc. (A), 1969, 372.

Moreover, other examples of the ruthenium compound, besides the ruthenium compounds mentioned above, include RuCl₃, Ru₃(CO)₁₂, RuCl₂(C₈H₁₂), Ru(CO)₃(C₈H₈), Ru(CO)₃(C₈H₁₂) and Ru(C₈H₁₀)(C₈H₁₂). These ruthenium compounds can also be used as precursor compounds to the ruthenium compounds mentioned above, and the above ruthenium compounds may be prepared and introduced into the reaction system either prior to or during the esterification reaction of the present invention.

Although there are no particular limitations on the amount used of the ruthenium compound, if due consideration is given to the production cost, then the amount is preferably as small as possible. However, from the viewpoint of achieving a practically applicable speed for the esterification reaction, the amount used of the ruthenium compound, relative to the norbornadiene used as one of the raw materials, is typically 1/10,000 equivalents or more, preferably 1/1,000 equivalents or more, and more preferably 1/100 equivalents or more. Further, from the viewpoint of achieving a reaction rate commensurate with the amount of the ruthenium compound, the amount used of the ruthenium compound relative to the norbornadiene is typically 1 equivalent or less, preferably 1/10 equivalents or less, and is more preferably 1/20 equivalents or less. In the present invention, a single ruthenium compound may be used alone, or a combination of a plurality of compounds may be used.

### (Cobalt Compound)

There are no particular limitations on the types of cobalt compounds that can be used in the present invention, provided the compound contains cobalt. Specific examples of preferred compounds include cobalt complex compounds having carbonyl ligands such as Co₂(CO)₈, HCo(CO)₄ and Co₄(CO)₁₂, cobalt complex compounds having a carboxylic acid ligand such as cobalt acetate, cobalt propionate, cobalt benzoate and cobalt citrate, and cobalt phosphate.

Although there are no particular limitations on the amount used of the cobalt compound, the amount of the cobalt compound relative to the amount of the ruthenium compound is typically 1/100 equivalents or more, preferably 1/10 equivalents or more, and more preferably ⅕ equivalents or more. Further, the amount of the cobalt compound relative to the amount of the ruthenium compound is typically 10 equivalents or less, preferably 5 equivalents or less, and more preferably 3 equivalents or less. The range described above is preferable from the viewpoint of maximizing the amount of the ester compound produced. In the present invention, a single cobalt compound may be used alone, or a combination of a plurality of compounds may be used.

### (Halide Salt)

There are no particular limitations on the types of halide salts that can be used in the present invention, provided the halide salt is a compound composed of a halide ion such as a chloride ion, a bromide ion or an iodide ion, and a cation. However, the halide salt used in the present invention is a salt that does not contain ruthenium and/or cobalt. The cation may be an inorganic ion or an organic ion. Further, the halide salt may contain one or more halide ions within the molecule.

The inorganic ion that constitutes the halide salt may be an ion of a metal selected from among alkali metals and alkaline earth metals. Specific examples of these metals include lithium, sodium, potassium, rubidium, cesium, calcium and strontium.

Further, the organic ion may be a monovalent or higher valency organic group derived from an organic compound. Examples include ammonium, phosphonium, pyrrolidinium, pyridium, imidazolium and iminium, and the hydrogen atoms within these ions may each be substituted with a hydrocarbon group such as an alkyl group or an aryl group. Although there are no particular limitations, specific examples of preferred organic ions include tetramethylammonium, tetraethylammonium, tetrapropylammonium, tetrabutylammonium, tetrapentylammonium, tetrahexylammonium, tetraheptylammonium, tetraoctylammonium, trioctylmethylammonium, benzyltrimethylammonium, benzyltriethylammonium, benzyltributylammonium, tetramethylphosphonium, tetraethylphosphonium, tetraphenylphosphonium, benzyltriphenylphosphonium and bis(triphenylphosphine)iminium.

The halide salt used in the present invention need not necessarily be a solid salt. An ionic liquid containing halide ions that becomes a liquid near room temperature or at a temperature of 100° C. or less may also be used as the halide salt. Specific examples of the cation used in this type of ionic liquid include an organic ions such as 1-ethyl-3-methylimidazolium, 1-propyl-3-methylimidazolium, 1-butyl-3-methylimidazolium, 1-pentyl-3-methylimidazolium, 1-hexyl-3-methylimidazolium, 1-heptyl-3-methylimidazolium, 1-octyl-3-methylimidazolium, 1-decyl-3-methylimidazolium, 1-dodecyl-3-methylimidazolium, 1-tetradecyl-3-methylimidazolium, 1-hexadecyl-3-methylimidazolium, 1-octadecyl-3-methylimidazolium, 1-ethyl-2,3-dimethylimidazolium, 1-butyl-2,3-dimethylimidazolium, 1-hexyl-2,3-dimethylimidazolium, 1-ethylpyridinium, 1-butylpyridinium, 1-hexylpyridinium, butylmethylpyrrolidinium, 8-methyl-1,8-diazabicyclo[5.4.0]-7-undecene, 8-ethyl-1,8-diazabicyclo[5.4.0]-7-undecene, 8-propyl-1,8-diazabicyclo[5.4.0]-7-undecene, 8-butyl-1,8-diazabicyclo[5.4.0]-7-undecene, 8-pentyl-1,8-diazabicyclo[5.4.0]-7-undecene, 8-hexyl-1,8-diazabicyclo[5.4.0]-7-undecene, 8-heptyl-1,8-diazabicyclo[5.4.0]-7-undecene and 8-octyl-1,8-diazabicyclo[5.4.0]-7-undecene.

Among the halide salts described above, preferred halide salts are compounds which are chloride salts, bromide salts or iodide salts, and in which the cation is an organic ion. Further, from the viewpoint of improving the reactivity, a quaternary ammonium salt is preferable. Quaternary ammonium salts also include compounds in which the substituent groups on the nitrogen atom are bonded to each other to form cyclic structures, and compounds in which one or more substituents are bonded to the nitrogen atom via a double bond. Although there are no particular limitations, specific examples of preferred halide salts in the present invention include butylmethylpyrrolidinium chloride, bis(triphenylphosphine)iminium iodide, trioctylmethylammonium chloride and tetraethylammonium chloride.

Although there are no particular limitations on the amount used of the halide salt, the amount of the halide salt relative to the amount of the ruthenium compound is typically 1 equivalent or more, preferably 1.5 equivalents or more, and more preferably 2 equivalents or more. When the amount of the halide salt satisfies this range, the reaction rate can be increased effectively. Further, the amount of the halide salt relative to the amount of the ruthenium compound is typically 1,000 equivalents or less, preferably 50 equivalents or less, and more preferably 10 equivalents or less. This range is preferred from the viewpoint of achieving an improvement in the reaction rate commensurate with the amount used. In the present invention, a single halide salt may be used alone, or a combination of a plurality of salts may be used.

### (Basic Compound)

The types of basic compounds that can be used in the present invention include both inorganic compounds and organic compounds. Specific examples of the basic inorganic compounds include carbonates, hydrogen carbonates, hydroxides and alkoxides of the various metals of the alkali metals and alkaline earth metals. Specific examples of the basic organic compounds include primary amine compounds, secondary amine compounds and tertiary amine compounds. Among the basic compounds mentioned above, tertiary amine compounds are preferred from the viewpoint of their effect in accelerating the reaction. The tertiary amine compounds also include compounds in which the substituent groups on the nitrogen atom are bonded to each other to form cyclic structures, and compounds in which a substituent is bonded to the nitrogen atom via a double bond. Accordingly, the tertiary amine compounds include pyridine compounds, imidazole compounds, and quinoline compounds and the like. Specific examples of preferred tertiary amine compounds in the present invention include trialkylamines, N-alkylpyrrolidines, N-alkylpiperidines, quinuclidine and triethylenediamine. Each of the alkyl groups in these compounds is preferably an alkyl group of 1 to 12 carbon atoms, and specific examples include a methyl group, ethyl group, propyl group, butyl group, pentyl group, hexyl group, heptyl group, octyl group, nonyl group, decyl group, undecyl group and dodecyl group, wherein these groups may be linear, branched or cyclic. In a trialkylamine, the three alkyl groups may be the same or different.

Although there are no particular limitations on the amount used of the basic compound, the amount of the basic compound relative to the amount of the ruthenium compound is typically 1 equivalent or more, preferably 2 equivalents or more, and more preferably 5 equivalents or more. When the amount of the basic compound satisfies this range, the effect of the basic compound in accelerating the reaction tends to be more dramatic. Further, the amount of the basic compound is typically 1,000 equivalents or less, preferably 200 equivalents or less, and more preferably 30 equivalents or less. This range is preferred from the viewpoint of achieving an improvement in the reaction rate commensurate with the amount used. In the present invention, a single basic compound may be used alone, or a combination of a plurality of compounds may be used.

In the production method according to the present invention, by adding, as required, one or both of a phenol compound and an organohalogen compound to the catalyst system containing the ruthenium compound, the cobalt compound, the halide salt and the basic compound, the effect of the catalyst system in accelerating the reaction can be further enhanced. Each of these compounds is described below.

### (Phenol Compound)

Specific examples of preferred phenol compounds for use in the present invention include phenol, cresols, alkylphenols, alkoxyphenols, phenoxyphenols, chlorophenols, trifluoromethylphenols, hydroquinone and catechol. The alkyl group in the alkylphenols and alkoxyphenols is preferably an alkyl group of 1 to 12 carbon atoms, and specific examples include a methyl group, ethyl group, propyl group, butyl group, pentyl group, hexyl group, heptyl group, octyl group, nonyl group, decyl group, undecyl group and dodecyl group, wherein these groups may be linear, branched or cyclic.

Although there are no particular limitations on the amount added of the phenol compound, the amount of the phenol compound relative to the amount of the ruthenium compound is typically 1 equivalent or more, preferably 2 equivalents or more, and more preferably 3 equivalents or more. When the amount added of the phenol compound satisfies this range, the effect of the phenol compound in accelerating the reaction tends to be more dramatic. Further, the amount of the phenol compound is typically 1,000 equivalents or less, preferably 50 equivalents or less, and more preferably 10 equivalents or less. This range is preferred from the viewpoint of achieving an improvement in the reaction rate commensurate with the amount added. In the present invention, a single phenol compound may be used alone, or a combination of a plurality of compounds may be used.

### (Organohalogen Compound)

Examples of preferred organohalogen compounds for use in the present invention include halogen-substituted aliphatic hydrocarbons and halogen-substituted aromatic hydrocarbons. Examples include alkyl halides such as methyl halides and ethyl halides, alkanes substituted with two or more halogens such as dihalogenomethanes, dihalogenoethanes, trihalogenomethanes and carbon tetrahalogens, and halogenated benzenes. Examples of the halogen include chlorine, bromine and iodine.

Although there are no particular limitations on the amount added of the organohalogen compound, the amount of the organohalogen compound relative to the amount of the ruthenium compound is typically 1 equivalent or more, preferably 2 equivalents or more, and more preferably 3 equivalents or more. When the amount added of the organohalogen compound satisfies this range, the effect of the organohalogen compound in accelerating the reaction tends to be more dramatic. Further, the amount of the organohalogen compound is typically 1,000 equivalents or less, preferably 50 equivalents or less, and more preferably 10 equivalents or less. This range is preferred from the viewpoint of achieving an improvement in the reaction rate commensurate with the amount added. In the present invention, a single organohalogen compound may be used alone, or a combination of a plurality of compounds may be used.

Moreover, a halogen-substituted phenol compound such as a chlorophenol or a trifluoromethylphenol can also be used as the phenol compound and the organohalogen compound. In this case, the amount added of the halogen-substituted phenol compound is preferably the same as the amount described above for the phenol compound or the organohalogen compound.

### (Solvent)

In the production method of the present invention, the reaction between the norbornadiene and the formic acid ester can proceed even without using a solvent. However, a solvent may be used if required. There are no particular limitations on the types of solvents that can be used in the present invention, provided the solvent is capable of dissolving the compounds used as raw materials. Specific examples of solvents that can be used favorably in the present invention include n-pentane, n-hexane, n-heptane, cyclohexane, benzene, toluene, o-xylene, p-xylene, m-xylene, ethylbenzene, cumene, tetrahydrofuran, N-methylpyrrolidone, dimethylformamide, dimethylacetamide, dimethylimidazolidinone, ethylene glycol dimethyl ether, diethylene glycol dimethyl ether, triethylene glycol dimethyl ether, and acetonitrile. When a solvent is used, either a single solvent may be used alone, or a combination of a plurality of solvents may be used.

### (Raw Material Ratio)

The ratio between the norbornadiene and the formic acid ester used in the reaction, in terms of the amounts added of each component, preferably provides 2 mol or more, and more preferably 4 mol or more of the formic acid ester, per 1 mol of the norbornadiene. When the ratio satisfies this range, side reactions can be suppressed, and a satisfactory yield tends to be obtainable. Further, the ratio between the norbornadiene and the formic acid ester, in terms of the amounts added of each component, preferably provides 100 mol or less, and more preferably 50 mol or less of the formic acid ester, per 1 mol of the norbornadiene. This range is preferable from the viewpoint of productivity.

### (Reaction Temperature)

In the production method of the present invention, the reaction between the norbornadiene and the formic acid ester is preferably performed within a temperature range from 80° C. to 200° C. The reaction is more preferably performed within a temperature range from 100° C. to 160° C. By performing the reaction at a temperature of 80° C. or more, the reaction rate is increased, and the reaction is able to proceed with good efficiency. On the other hand, by restricting the reaction temperature to 200° C. or less, decomposition of the formic acid ester used as a raw material can be suppressed. If the formic acid ester decomposes, then addition of ester groups to the norbornadiene becomes unachievable. Moreover, if the reaction temperature is too high, then ring-opening polymerization of the norbornadiene raw material can occur, and there is a chance that the yield may decrease. In those cases where the reaction temperature exceeds the boiling point of either the norbornadiene or the formic acid ester used as raw materials, the reaction is preferably conducted inside a pressure-resistant container. The end of the reaction can be confirmed using conventional analysis techniques such as gas chromatography or NMR or the like.

By using the production method described above, a norbornanedicarboxylic acid ester having a high exo isomer content can be obtained with good efficiency. According to an embodiment of the present invention, a norbornanedicarboxylic acid ester can be obtained which has an exo isomer content (exo isomer (mol)/(exo isomer+endo isomer (mol)) of 60% or more, preferably 65% or more, and more preferably 70% or more.

Further, according to an embodiment of the present invention, a norbornanedicarboxylic acid ester can be obtained with a high yield, for example a yield based on the norbornadiene (norbornanedicarboxylic acid ester (mol)/norbornadiene (mol)) of 50% or more, preferably 55% or more, and more preferably 60% or more.

In the present invention, by subsequently separating the obtained norbornanedicarboxylic acid ester into the endo-norbornanedicarboxylic acid ester and the exo-norbornanedicarboxylic acid ester, the exo-norbornanedicarboxylic acid ester can be obtained.

Examples of embodiments of the exo-norbornanedicarboxylic acid ester include exo-norbornanedicarboxylic acid esters represented by a formula (V) or a formula (VI) shown below.

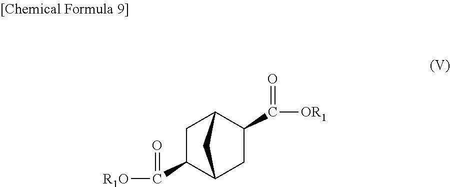

(In the formula, each R₁ independently represents an alkyl group of 1 to 5 carbon atoms, a vinyl group, or a benzyl group.)

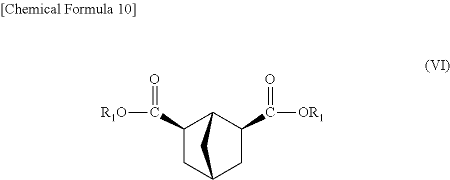

(In the formula, each R₁ independently represents an alkyl group of 1 to 5 carbon atoms, a vinyl group, or a benzyl group.)

Examples of methods that can be used for separating the norbornanedicarboxylic acid ester (exo/endo mixture) into the endo-norbornanedicarboxylic acid ester and the exo-norbornanedicarboxylic acid ester include conventional methods such as reduced-pressure distillation and recrystallization.

Furthermore, in the present invention, a norbornanedicarboxylic acid can be obtained from the norbornanedicarboxylic acid ester. Examples of methods that can be used for obtaining the norbornanedicarboxylic acid from the norbornanedicarboxylic acid ester include conventional hydrolysis methods such as treatment with an acid or an alkali.

### EXAMPLES

The present invention is described below in further detail based on a series of examples. However, the scope of the present invention is in no way limited by these examples.

### Example 1

A stainless steel pressure reaction apparatus having an internal capacity of 50 ml was charged, at room temperature, with 0.05 mmol of [Ru(CO)₃Cl₂]₂ as the ruthenium compound ( 1/50 equivalents relative to the norbornadiene), 0.05 mmol of Co₂(CO)₈ as the cobalt compound (1 equivalent relative to the ruthenium compound), 0.25 mmol of butylmethylpyrrolidinium chloride as the halide salt (5 equivalents relative to the ruthenium compound), and 0.5 mmol of triethylamine as the basic compound (10 equivalents relative to the ruthenium compound), and the compounds were mixed to obtain a catalyst system. To this catalyst system were added 2.5 mmol of norbornadiene (manufactured by Tokyo Chemical Industry Co., Ltd.) and 5.0 ml of methyl formate (manufactured by Mitsubishi Gas Chemical Company, Inc.) (32.9 mol per 1 mol of norbornadiene), and the inside of the reaction apparatus was then purged with nitrogen gas at 0.5 MPa, and then held at 120° C. for 15 hours. Subsequently, the reaction apparatus was cooled to room temperature, the pressure was released, a portion of the residual organic phase was removed, and the components of the reaction mixture were analyzed using a gas chromatograph under the conditions described below. The analysis results revealed that the norbornanedicarboxylic acid methyl ester produced by the reaction was obtained in an amount of 1.23 mmol (a yield of 49.2% based on the norbornadiene), and the exo/endo composition ratio (molar ratio) was 75/25. Further, in this case, the exo isomer and the endo isomer each exhibited two peaks in the gas chromatograph, and therefore it is assumed that both the 2,5-isomer and the 2,6-isomer were produced. The gas chromatograph analysis was conducted under the following conditions, using a GC-353B-model GC manufactured by GL Sciences Inc.

Detector: Hydrogen flame ionization detector

Column: TC-1 (60 m) manufactured by GL Sciences Inc.

Carrier gas: Helium (300 kPa)

Temperatures:
Injection port: 200° C.
Detector: 200° C.
Column: 40° C. to 240° C. (rate of temperature increase: 5° C./min)

### Comparative Example 1

### Catalyst System Containing Only the Ruthenium Compound and the Halide Salt

With the exception of not using the cobalt compound and the basic compound from the catalyst system of Example 1, reaction was performed under exactly the same conditions as Example 1. When the obtained reaction mixture was analyzed in the same manner as that described for Example 1, the amount of norbornanedicarboxylic acid methyl ester produced by the reaction was only a trace amount.

### Comparative Example 2

### Catalyst System Containing Only the Cobalt Compound and the Halide Salt

With the exception of not using the ruthenium compound and the basic compound from the catalyst system of Example 1, reaction was performed under exactly the same conditions as Example 1. When the components of the obtained reaction mixture were analyzed by gas chromatography, the amount of norbornanedicarboxylic acid methyl ester produced by the reaction was only a trace amount.

### Comparative Example 3

### Catalyst System Containing Only the Ruthenium Compound and the Cobalt Compound

With the exception of not using the halide salt and the basic compound from the catalyst system of Example 1, reaction was performed under exactly the same conditions as Example 1. When the obtained reaction mixture was analyzed by gas chromatography, the amount of norbornanedicarboxylic acid methyl ester produced by the reaction was only a trace amount.

### Comparative Example 4

### Catalyst System Containing Only the Ruthenium Compound, the Cobalt Compound and the Halide Salt

With the exception of not using the basic compound from the catalyst system of Example 1, reaction was performed under exactly the same conditions as Example 1. When the obtained reaction mixture was analyzed by gas chromatography, the amount of norbornanedicarboxylic acid methyl ester produced by the reaction was only a trace amount.

### Example 2

With the exception of using 0.5 mmol of tripropylamine as the basic compound in the catalyst system of Example 1, operations were performed in exactly the same manner as Example 1. The amount of the norbornanedicarboxylic acid methyl ester produced by the reaction was 0.83 mmol (a yield of 33.2% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

### Example 3

With the exception of using 0.5 mmol of N-methylpyrrolidine as the basic compound in the catalyst system of Example 1, operations were performed in exactly the same manner as Example 1. The amount of the norbornanedicarboxylic acid methyl ester produced by the reaction was 1.33 mmol (a yield of 53.2% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

### Example 4

With the exception of using 1.0 mmol of triethylamine as the basic compound (20 equivalents relative to the ruthenium compound) in the catalyst system of Example 1, operations were performed in exactly the same manner as Example 1. The amount of the norbornanedicarboxylic acid methyl ester produced by the reaction was 1.63 mmol (a yield of 65.2% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

Next, the exo isomer mentioned above (having two peaks in the gas chromatograph) was separated by distillation under reduced pressure.

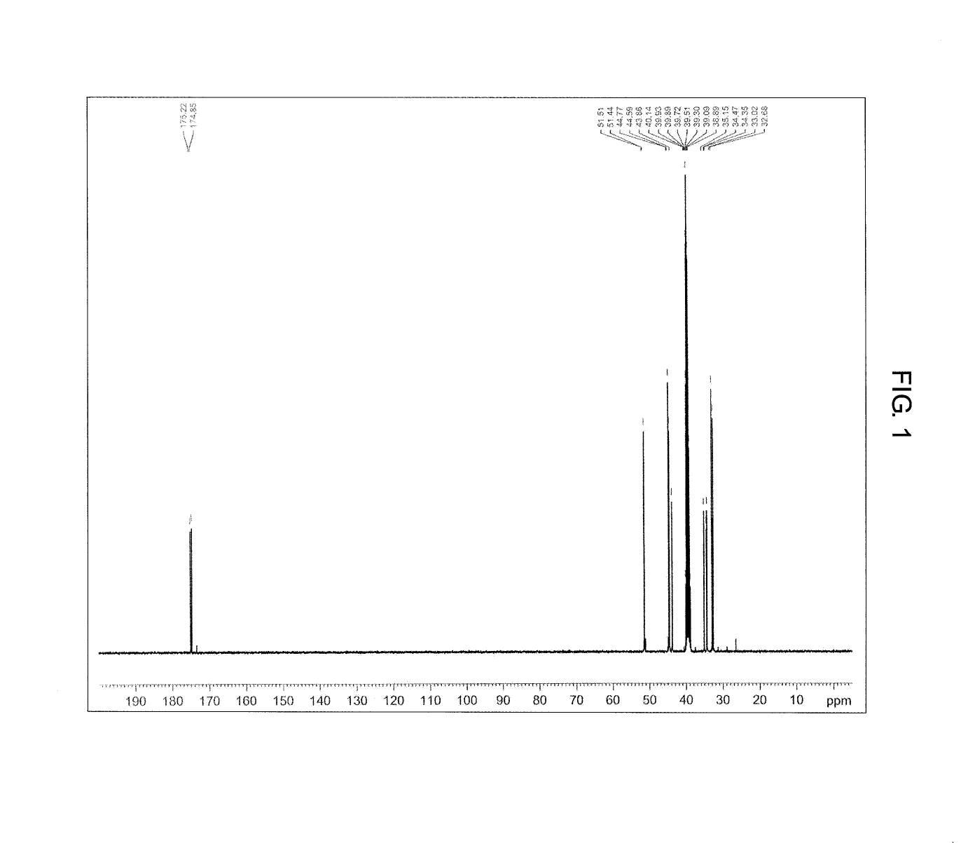

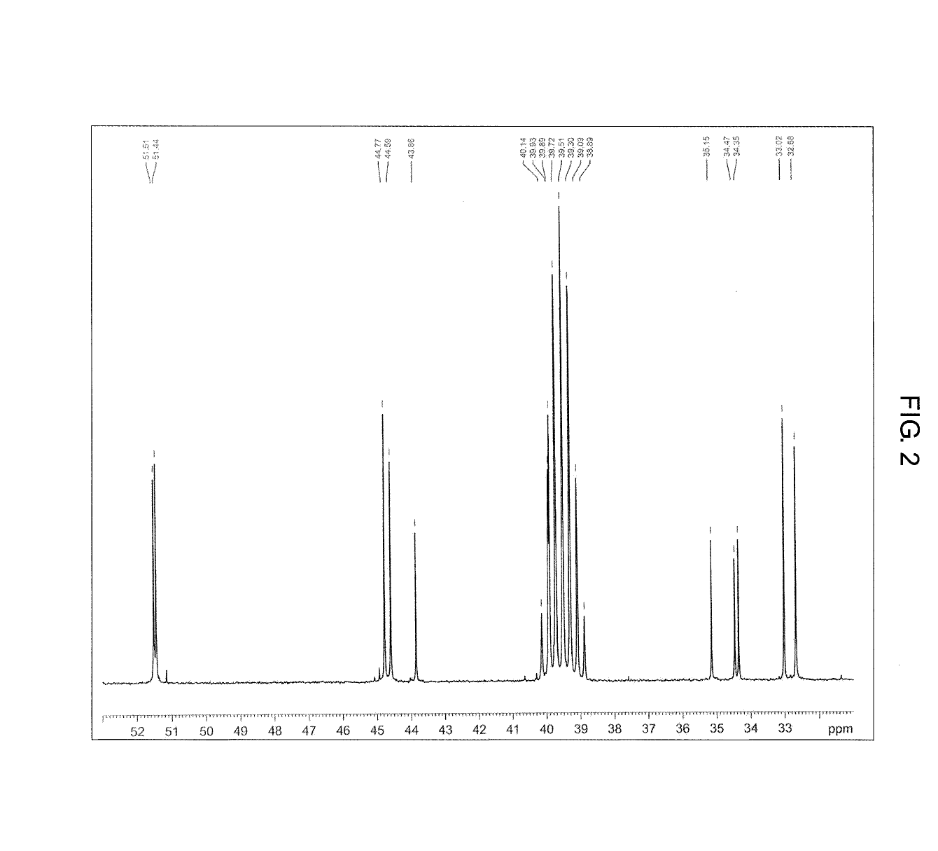

The ¹³C-NMR spectrum of the thus obtained exo-norbornanedicarboxylic acid methyl ester is illustrated in FIG. 1 and FIG. 2. The measurement conditions and identification data for the ¹³C-NMR spectrum were as follows.

Conditions: solvent DMSO-d6, apparatus AV400M manufactured by Bruker Corporation (carbon fundamental frequency: 100.62 MHz)

The results of ¹³C-NMR analysis revealed carbonyl carbons in the vicinity of 170 to 180 ppm, methyl ester carbons in the vicinity of 51 to 52 ppm, methylene carbons in the vicinity of 32 to 35 ppm, and methine carbons in the vicinity of 36 to 45 ppm. The number of each of these types of carbon atoms was carbonyl/methyl ester/methylene/methine=2/2/4/5. Each of these carbons was assigned as shown below.

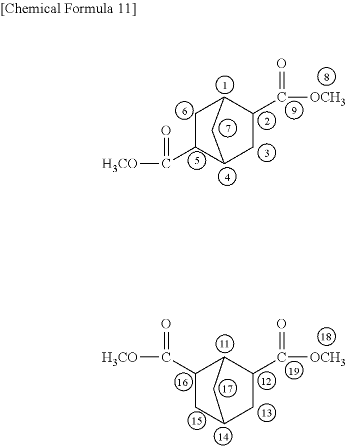

Carbon (1): 39.89 ppm peak (methine)

Carbon (2): 44.59 ppm peak (methine)

Carbon (3): 33.03 ppm peak (methylene)

Carbon (4): 39.89 ppm peak (methine)

Carbon (5): 44.59 ppm peak (methine)

Carbon (6): 33.02 ppm peak (methylene)

Carbon (7): 34.35 ppm peak (methylene)

Carbon (8): 51.44 ppm peak (methyl ester)

Carbon (9): 175.22 ppm peak (carbonyl)

Carbon (11): 35.15 ppm peak (methine)

Carbon (12): 44.77 ppm peak (methine)

Carbon (13): 32.68 ppm peak (methylene)

Carbon (14): 43.86 ppm peak (methine)

Carbon (15): 32.68 ppm peak (methylene)

Carbon (16): 44.77 ppm peak (methine)

Carbon (17): 34.47 ppm peak (methylene)

Carbon (18): 51.51 ppm peak (methyl ester)

Carbon (19): 174.85 ppm peak (carbonyl)

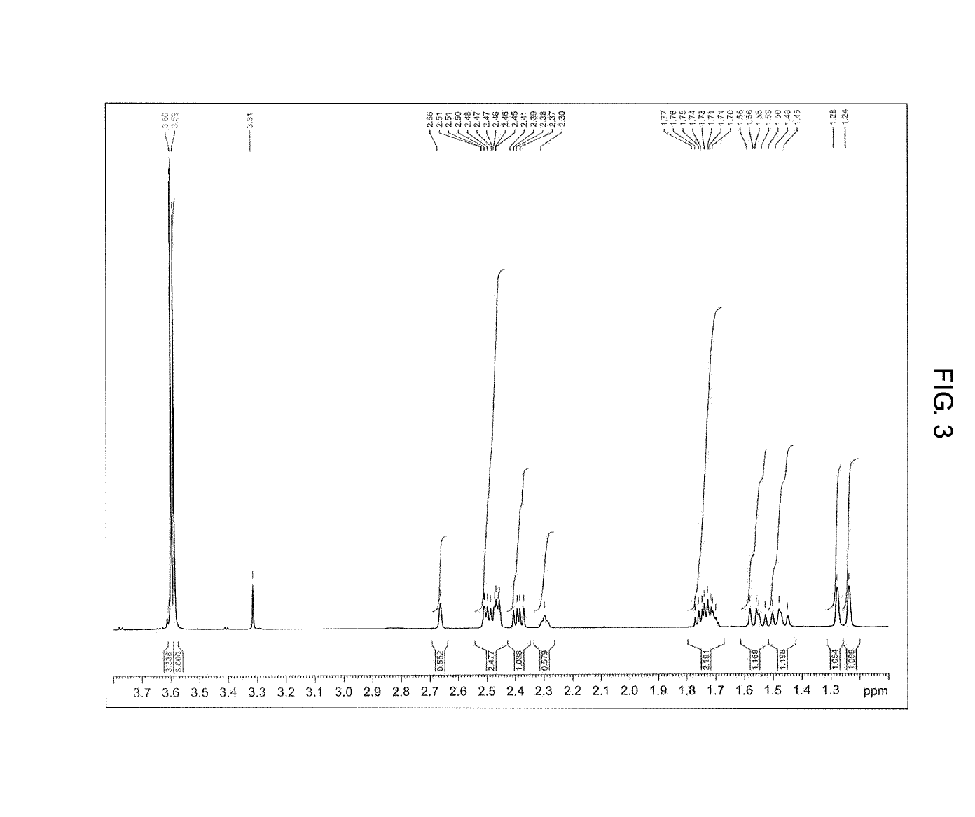

The ¹H-NMR spectrum of the thus obtained exo-norbornanedicarboxylic acid methyl ester is illustrated in FIG. 3. The measurement conditions and identification data for the ¹H-NMR spectrum were as follows.

Conditions: solvent DMSO-d6, apparatus AV400M manufactured by Bruker Corporation (proton fundamental frequency: 400.13 MHz)

As a result of the ¹H-NMR analysis, each of the protons was assigned as shown below.

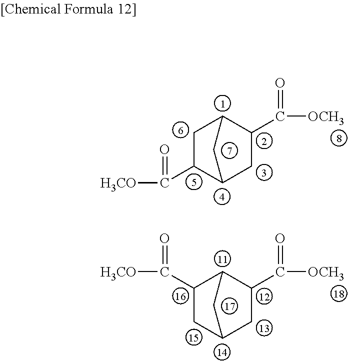 [SMILES: COC(=O)C1CC2CC1CC2C(=O)OC ; COC(=O)C1CC2CC(C(=O)OC)C1C2]

Proton (1): peak in the vicinity of 2.47 ppm (methine)

Proton (2): peak in the vicinity of 2.4 ppm (methine)

Proton (3): peak in the vicinity of 1.5 ppm to 1.8 ppm (methylene)

Proton (4): peak in the vicinity of 2.47 ppm (methine)

Proton (5): peak in the vicinity of 2.4 ppm (methine)

Proton (6): peak in the vicinity of 1.5 ppm to 1.8 ppm (methylene)

Proton (7): peak in the vicinity of 1.3 ppm (methylene)

Proton (8): peak in the vicinity of 3.6 ppm (methyl)

Proton (11): peak in the vicinity of 2.3 ppm (methine)

Proton (12): peak in the vicinity of 2.5 ppm (methine)

Proton (13): peak in the vicinity of 1.5 ppm to 1.8 ppm (methylene)

Proton (14): peak in the vicinity of 2.7 ppm (methine)

Proton (15): peak in the vicinity of 1.5 ppm to 1.8 ppm (methylene)

Proton (16): peak in the vicinity of 2.5 ppm (methine)

Proton (17): peak in the vicinity of 1.2 ppm (methylene)

Proton (18): peak in the vicinity of 3.6 ppm (methyl)

Further, based on the integral intensity ratios, it was confirmed that 4 methyl groups, 6 methylene groups and 8 methine groups existed in the compound.

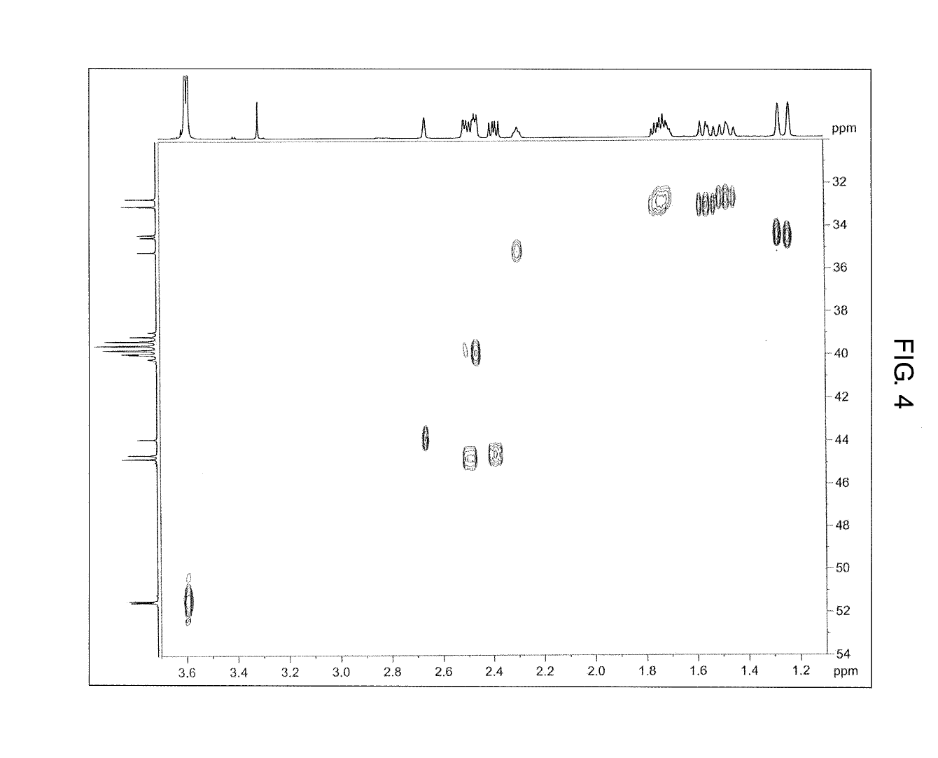

The ¹H-¹³C HSQC spectrum of the thus obtained exo-norbornanedicarboxylic acid methyl ester is illustrated in FIG. 4. Based on the ¹H-¹³C HSQC spectrum, correlations were confirmed between the carbons and protons having the same peak numbers mentioned above, thus confirming that the peak assignments made in FIG. 1, FIG. 2 and FIG. 3 were correct.

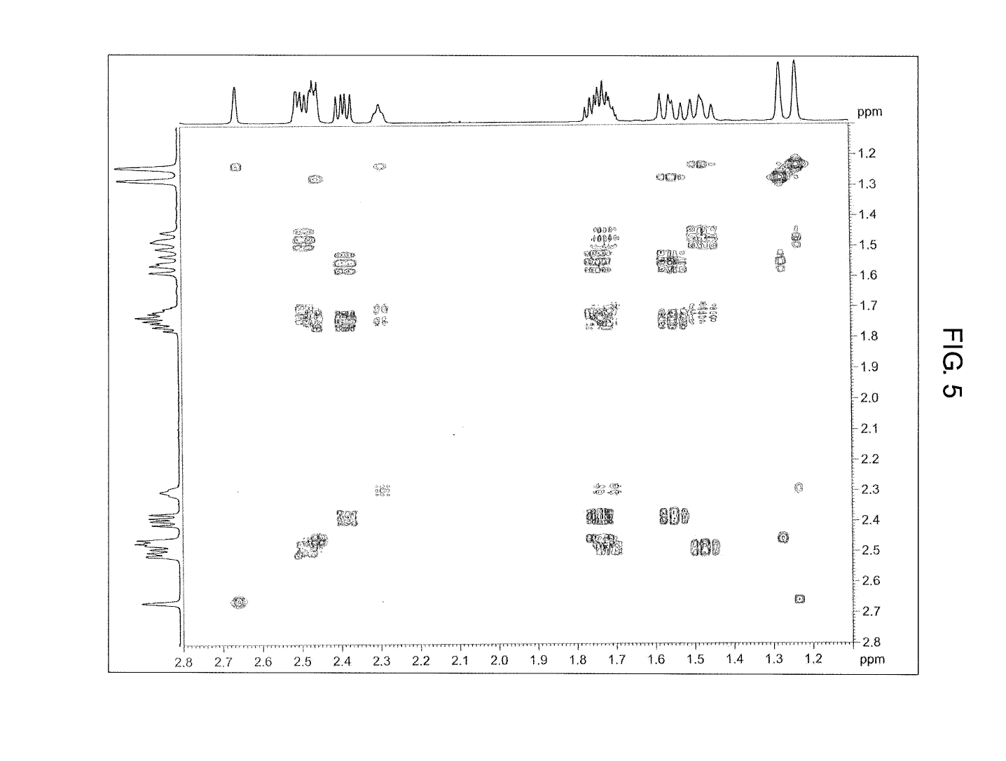

The ¹H-¹H COSY spectrum of the thus obtained exo-norbornanedicarboxylic acid methyl ester is illustrated in FIG. 5. FIG. 5 reveals correlations between protons (1) and (4) and proton (7), between protons (1) and (4) and protons (3) and (6), between protons (2) and (5) and protons (3) and (6), between protons (11) and (14) and proton (17), between protons (12) and (16) and protons (13) and (15), and between protons (13) and (15) and proton (14), confirming that protons (1) to (7) and protons (11) to (17) respectively constitute a norbornane ring.

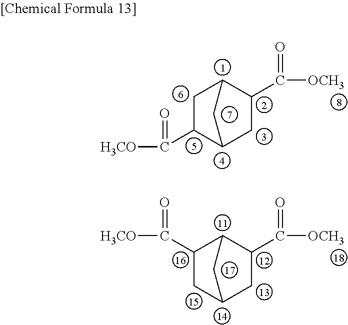 [SMILES: COC(=O)C1CC2CC1CC2C(=O)OC ; COC(=O)C1CC2CC(C(=O)OC)C1C2]

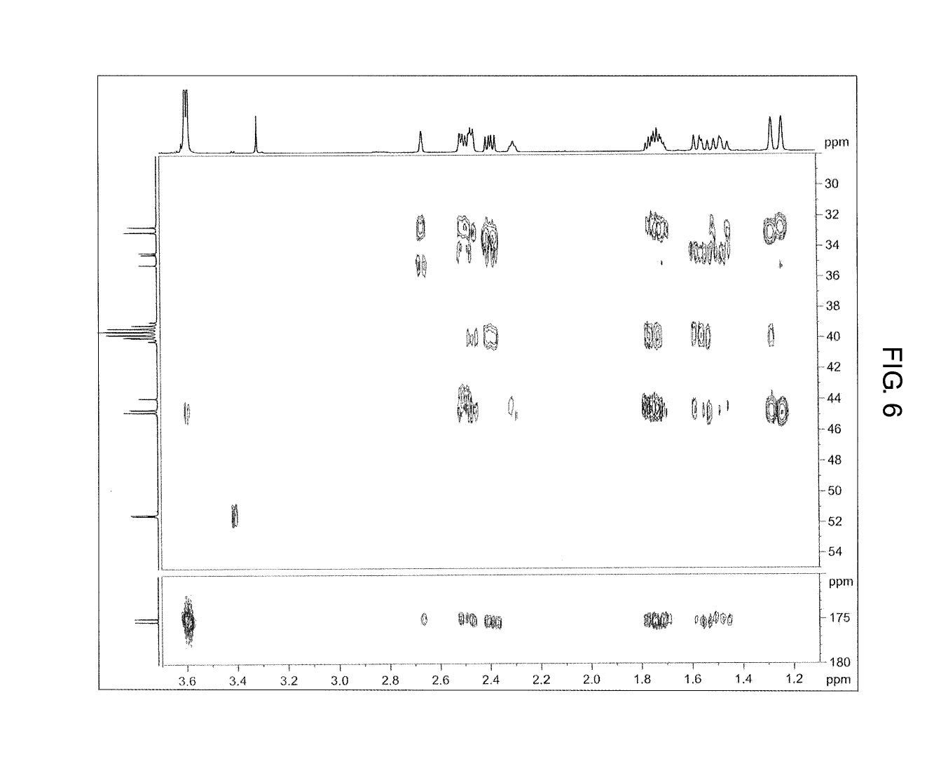

The ¹H-¹³C HMBC spectrum of the thus obtained exo-norbornanedicarboxylic acid methyl ester is illustrated in FIG. 6. The ¹H-¹³C HMBC spectrum confirmed the structural identification of the two compounds.

(1) Compound in which the Norbornane Ring is Composed of Protons (1) to (7)

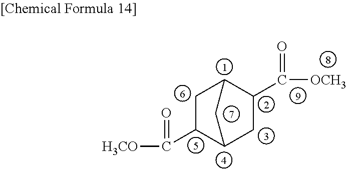

From FIG. 6, correlations were confirmed between the carbonyl carbon (9) and the methine proton (2) and the methine proton (5), thus confirming the compound as norbornane-2,5-dicarboxylic acid methyl ester.

(2) Compound in which the Norbornane Ring is Composed of Protons (11) to (17)

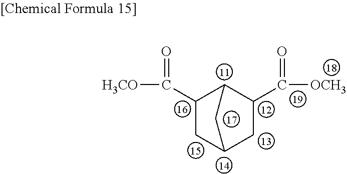

From FIG. 6, correlations were confirmed between the carbonyl carbon (19) and the methine proton (12) and the methine proton (16), thus confirming the compound as norbornane-2,6-dicarboxylic acid methyl ester.

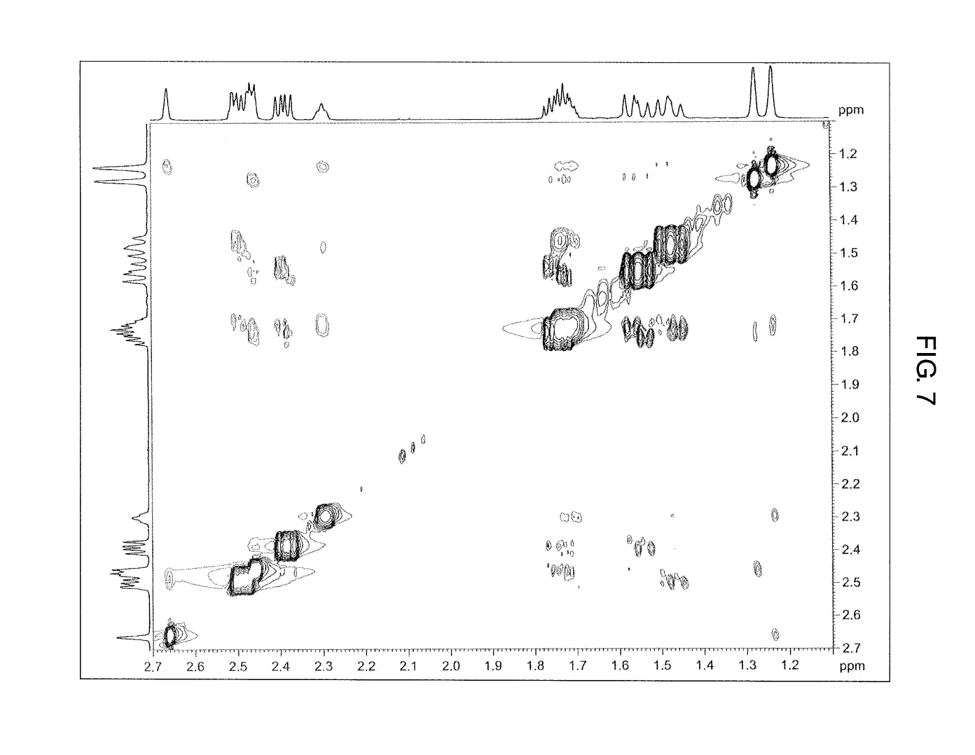

The ¹H-¹H NOESY spectrum of the thus obtained exo-norbornanedicarboxylic acid methyl ester is illustrated in FIG. 7. The ¹H-¹H NOESY spectrum confirmed the isomeric structural identifications of norbornane-2,5-dicarboxylic acid methyl ester and norbornane-2,6-dicarboxylic acid methyl ester.

### (1) Norbornane-2,5-Dicarboxylic Acid Methyl Ester

From FIG. 7, a correlation exists between the protons (1) and (4) and the proton (7), but no correlation was observed with the protons (2) and (5), confirming that the protons (2) and (5) are bonded in the endo-positions. Accordingly, it was confirmed that this compound was norbornane-2(exo)-5(exo)-dicarboxylic acid methyl ester.

(2) Norbornane-2,6-dicarboxylic Acid Methyl Ester

From FIG. 7, a correlation exists between the protons (11) and (14) and the proton (17), but no correlation was observed with the protons (12) and (16), confirming that the protons (12) and (16) are bonded in the endo-positions. Accordingly, it was confirmed that this compound was norbornane-2(exo)-6(exo)-dicarboxylic acid methyl ester.

### Example 5

With the exception of adding 0.25 mmol of p-cresol as a phenol compound (5 equivalents relative to the ruthenium compound) to the catalyst system of Example 4, operations were performed in exactly the same manner as Example 4. The amount of the norbornanedicarboxylic acid methyl ester produced by the reaction was 1.74 mmol (a yield of 69.6% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

TABLE 1

| | Composition (mmol) | | | | | Reaction results | |
|---|---|---|---|---|---|---|---|
| | Ruthenium | Cobalt | Halide | Basic | Phenol | Yield | Exo/endo |
| Item | compound | compound | salt | compound | compound | (%) | ratio |
| Example 1 | [Ru(CO)₃Cl₂]₂ | Co₂(CO)₈ | [bmpy]Cl | TEA | — | 49.2 | 75/25 |
| | 0.05 | 0.05 | 0.25 | 0.5 | | | |
| Example 2 | [Ru(CO)₃Cl₂]₂ | Co₂(CO)₈ | [bmpy]Cl | TPA | — | 33.2 | 75/25 |
| | 0.05 | 0.05 | 0.25 | 0.5 | | | |
| Example 3 | [Ru(CO)₃Cl₂]₂ | Co₂(CO)₈ | [bmpy]Cl | N-methyl | — | 53.2 | 75/25 |
| | 0.05 | 0.05 | 0.25 | pyrrolidine | | | |
| | | | | 0.5 | | | |
| Example 4 | [Ru(CO)₃Cl₂]₂ | Co₂(CO)₈ | [bmpy]Cl | TEA | — | 65.2 | 75/25 |
| | 0.05 | 0.05 | 0.25 | 1.0 | | | |
| Example 5 | [Ru(CO)₃Cl₂]₂ | Co₂(CO)₈ | [bmpy]Cl | TEA | p-Cresol | 69.6 | 75/25 |
| | 0.05 | 0.05 | 0.25 | 1.0 | 0.25 | | |
| Comparative | [Ru(CO)₃Cl₂]₂ | — | [bmpy]Cl | — | — | trace | — |
| Example 1 | 0.05 | | 0.25 | | | | |
| Comparative | — | Co₂(CO)₈ | [bmpy]Cl | — | — | trace | — |
| Example 2 | | 0.05 | 0.25 | | | | |
| Comparative | [Ru(CO)₃Cl₂]₂ | Co₂(CO)₈ | — | — | — | trace | — |
| Example 3 | 0.05 | 0.05 | | | | | |
| Comparative | [Ru(CO)₃Cl₂]₂ | Co₂(CO)₈ | [bmpy]Cl | — | — | trace | — |
| Example 4 | 0.05 | 0.05 | 0.25 | | | | |

The results of Examples 1 to 5 and Comparative Examples 1 to 4 are shown in Table 1. In the present invention, by performing the esterification reaction in the presence of the ruthenium compound, the cobalt compound, the halide salt and the basic compound, a norbornanedicarboxylic acid ester having a high exo isomer content can be obtained with good efficiency. Using a large amount of the basic compound, and using a phenol compound in addition to the ruthenium compound, the cobalt compound, the halide salt and the basic compound are effective in obtaining the norbornanedicarboxylic acid ester in even higher yield.

### Example 6

With the exceptions of using 0.25 mmol of trioctylmethylammonium chloride as the halide salt and 1.0 mmol of dimethylethylamine as the basic compound in the catalyst system of Example 4, operations were performed in exactly the same manner as Example 4. The amount of the norbornanedicarboxylic acid methyl ester produced by the reaction was 1.42 mmol (a yield of 56.8% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

### Example 7

With the exception of using 1.0 mmol of triethylamine as the basic compound in the catalyst system of Example 6, operations were performed in exactly the same manner as Example 6. The amount of the norbornanedicarboxylic acid methyl ester produced by the reaction was 1.32 mmol (a yield of 52.8% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

### Example 8

With the exception of using 0.05 mmol of cobalt citrate as the cobalt compound in the catalyst system of Example 7, operations were performed in exactly the same manner as Example 7. The amount of the norbornanedicarboxylic acid methyl ester produced by the reaction was 0.35 mmol (a yield of 14.0% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

### Example 9

With the exception of using 1.0 mmol of N,N-dimethylcyclohexylamine as the basic compound in the catalyst system of Example 7, operations were performed in exactly the same manner as Example 7. The amount of the norbornanedicarboxylic acid methyl ester produced by the reaction was 1.00 mmol (a yield of 40.0% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

TABLE 2

| | Composition (mmol) | | | | | Reaction results | |
|---|---|---|---|---|---|---|---|
| | Ruthenium | Cobalt | Halide | Basic | Phenol | Yield | Exo/endo |
| Item | compound | compound | salt | compound | compound | (%) | ratio |
| Example 6 | [Ru(CO)₃Cl₂]₂ | Co₂(CO)₈ | [toma]Cl | Me₂NEt | — | 56.6 | 75/25 |
| | 0.05 | 0.05 | 0.25 | 1.0 | | | |
| Example 7 | [Ru(CO)₃Cl₂]₂ | Co₂(CO)₈ | [toma]Cl | TEA | — | 52.8 | 75/25 |
| | 0.05 | 0.05 | 0.25 | 1.0 | | | |
| Example 8 | [Ru(CO)₃Cl₂]₂ | Co citrate | [toma]Cl | TEA | — | 14.0 | 75/25 |
| | 0.05 | 0.05 | 0.25 | 1.0 | | | |
| Example 9 | [Ru(CO)₃Cl₂]₂ | Co₂(CO)₈ | [toma]Cl | DMCHA | — | 40.0 | 75/25 |
| | 0.05 | 0.05 | 0.25 | 1.0 | | | |

The results of Examples 6 to 9 are shown in Table 2. Using a compound having carbonyl ligands as the cobalt compound was effective in obtaining the norbornanedicarboxylic acid ester in high yield. Moreover, as is evident by comparing Example 4 and Example 7, using an ionic liquid as the halide salt is also effective in achieving a high yield.

### Example 10

With the exception of using 0.05 mmol of [Ru(CO)₂Cl₂]ₙ, prepared in advance from ruthenium chloride and formic acid in accordance with the method disclosed in M. J. Cleare, W. P. Griffith, J. Chem. Soc. (A), 1969, 372, as the ruthenium compound in the catalyst system of Example 8, operations were performed in exactly the same manner as Example 8. The amount of the norbornanedicarboxylic acid methyl ester produced by the reaction was 1.13 mmol (a yield of 45.2% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

### Example 11

With the exception of using 0.25 mmol of tetraethylammonium chloride as the halide salt in the catalyst system of Example 10, operations were performed in exactly the same manner as Example 10. The amount of the norbornanedicarboxylic acid methyl ester produced by the reaction was 1.41 mmol (a yield of 56.4% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

### Example 12

With the exception of adding 0.25 mmol of hydroquinone monomethyl ether as a phenol compound to the catalyst system of Example 11, operations were performed in exactly the same manner as Example 11. The amount of the norbornanedicarboxylic acid methyl ester produced by the reaction was 1.65 mmol (a yield of 66.0% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

### Example 13

With the exception of using 0.25 mmol of cobalt acetate as the cobalt compound in the catalyst system of Example 11, operations were performed in exactly the same manner as Example 11. The amount of the norbornanedicarboxylic acid methyl ester produced by the reaction was 1.74 mmol (a yield of 69.6% based on the norbornadiene), and the exo/endo composition ratio was 75/25. Further, in this case, the exo isomer and the endo isomer each exhibited two peaks in the gas chromatograph, and therefore it is assumed that both the 2,5-isomer and the 2,6-isomer were produced.

TABLE 3

| | Composition (mmol) | | | | | Reaction results | |
|---|---|---|---|---|---|---|---|
| | Ruthenium | Cobalt | Halide | Basic | Phenol | Yield | Exo/endo |
| Item | compound | compound | salt | compound | compound | (%) | ratio |
| Example 10 | [Ru(CO)₂Cl₂]ₙ | Co citrate | [toma]Cl | TEA | — | 45.2 | 75/25 |
| | 0.05 | 0.05 | 0.25 | 1.0 | | | |
| Example 11 | [Ru(CO)₂Cl₂]ₙ | Co citrate | [tea]Cl | TEA | — | 56.4 | 75/25 |
| | 0.05 | 0.05 | 0.25 | 1.0 | | | |
| Example 12 | [Ru(CO)₂Cl₂]ₙ | Co citrate | [tea]Cl | TEA | MeHQ | 66.0 | 75/25 |
| | 0.05 | 0.05 | 0.25 | 1.0 | 0.25 | | |
| Example 13 | [Ru(CO)₂Cl₂]ₙ | Co acetate | [tea]Cl | TEA | — | 69.6 | 75/25 |
| | 0.05 | 0.05 | 0.25 | 1.0 | | | |

The results of Examples 10 to 13 are shown in Table 3. Using triethylammonium chloride as the basic halide salt, and using cobalt acetate as the cobalt compound are effective in obtaining the norbornanedicarboxylic acid ester in high yield. Moreover, as is evident by comparing Example 8 and Example 10, using [Ru(CO)₂Cl₂]ₙ as the ruthenium compound is also effective in achieving a high yield.

A description of the reference signs used in Tables 1 to 3, and the sources used for obtaining the catalyst systems is provided below.

[Ru(CO)₃Cl₂]₂: Strem Chemicals Inc.

Co₂(CO)₈: Tokyo Chemical Industry Co., Ltd.

Co citrate: cobalt citrate dihydrate, Alfa Aesar Ltd.

Co acetate: cobalt acetate tetrahydrate, Tokyo Chemical Industry Co., Ltd.

[bmpy]Cl: butylmethylpyrrolidinium chloride, Tokyo Chemical Industry Co., Ltd.

[toma]Cl: trioctylmethylammonium chloride, Tokyo Chemical Industry Co., Ltd.

[tea]Cl: tetraethylammonium chloride, Lion Corporation

TEA: triethylamine, Wako Pure Chemical Industries, Ltd.

TPA: tripropylamine, Tokyo Chemical Industry Co., Ltd.

N-methylpyrrolidine: Tokyo Chemical Industry Co., Ltd.

Me₂NEt: dimethylethylamine, Tokyo Chemical Industry Co., Ltd.

DMCHA: N,N-dimethylcyclohexylamine, Tokyo Chemical Industry Co., Ltd.

p-cresol: Wako Pure Chemical Industries, Ltd.

MeHQ: hydroquinone monomethyl ether, Kawaguchi Chemical Industry Co., Ltd.

### Reference Example 1

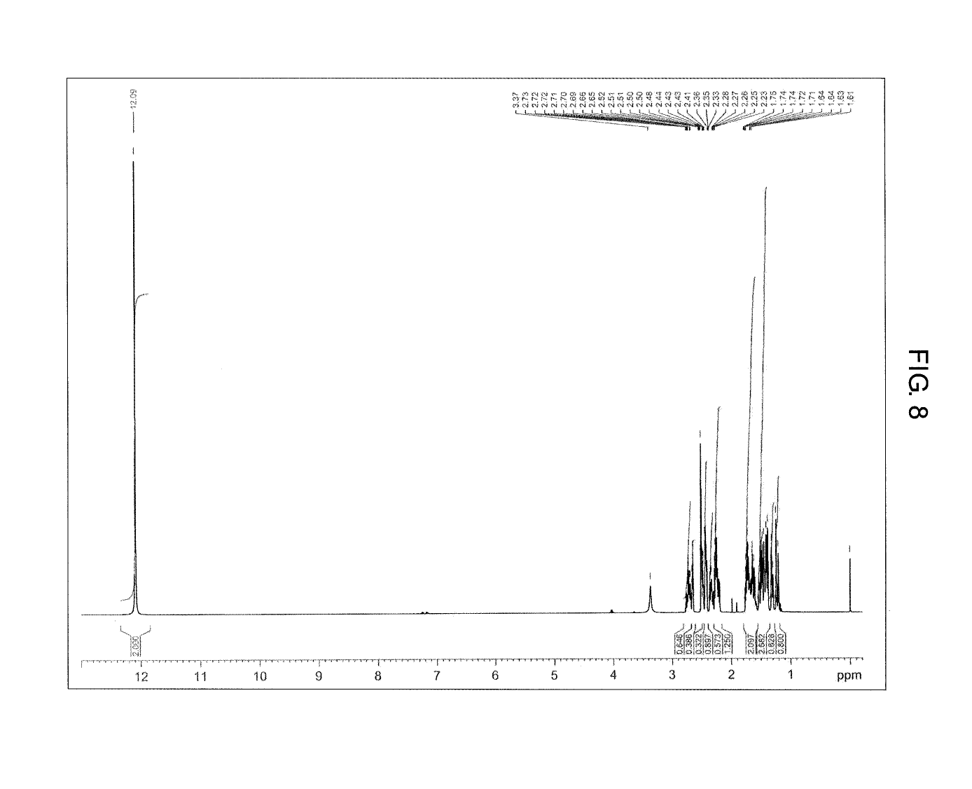

A 1 liter round-bottom flask fitted with a condenser tube was charged with 30 g of exo-norbornanedicarboxylic acid methyl ester obtained using the same method as that described in Example 4 and 200 g of methanol, and following uniform dissolution, 200 g of a 10% solution of sodium hydroxide was added, and the flask was placed in an oil bath at 100° C. and heated under reflux for 6 hours. Subsequently, sufficient methanol was removed by distillation to reduce the amount of the reaction liquid to 140 g, and when 48 ml of 36% hydrochloric acid was then added to the reaction mixture to adjust the pH to 1, a white powder precipitated. This white powder was collected by filtration, washed with water and dried, yielding 25 g of exo-norbornanedicarboxylic acid. The results of analyzing the thus obtained norbornanedicarboxylic acid by ¹H-NMR (FIG. 8) revealed peaks for the methylene and methine groups of the norbornane ring in the vicinity of 1.1 to 3.0 ppm, and a hydroxyl group peak attributed to the carboxylic acid in the vicinity of 12.4 ppm, and the integral intensity ratio between the peaks was 10.00/1.98 (theoretical value: 10/2).

As described above, the production method of the present invention enables a norbornanedicarboxylic acid ester having a high exo isomer content to be produced with good efficiency. The case in which methyl formate is used was presented as an example, but similar effects can be obtained when other formate esters are used.

### INDUSTRIAL APPLICABILITY

According to an embodiment of the present invention, a norbornanedicarboxylic acid ester having a high content of the desired exo isomer can be produced efficiently and in high yield, in a single step reaction, using inexpensive raw materials. The method according to an embodiment of the present invention can be achieved with minimal investment in equipment, and can suppress environmental impact to minimal levels, and therefore readily satisfies the needs of the industry.

Further, a polymer produced using the norbornanedicarboxylic acid ester having a high exo isomer content obtained in accordance with an embodiment of the present invention as a polymerization raw material exhibits excellent heat resistance, insulating properties, light resistance and mechanical properties, and can therefore be used for electronic components used in semiconductors and liquid crystals, for optical materials typified by optical fibers and optical lenses, and also as a material for display related applications and a material for medical purposes.

1. A method of producing a norbornanedicarboxylic acid ester, comprising a step of reacting a norbornadiene and a formic acid ester in the presence of a ruthenium compound, a cobalt compound, a halide salt and a basic compound.

2. The method of producing a norbornanedicarboxylic acid ester according to claim 1,
wherein the norbornanedicarboxylic acid ester is represented by a formula (I) or a formula (II) shown below: 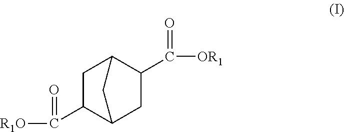
(wherein in the formula, each R₁ independently represents an alkyl group of 1 to 5 carbon atoms, a vinyl group, or a benzyl group) 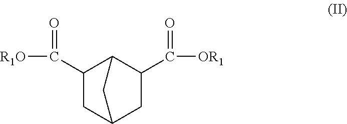
(wherein in the formula, each R₁ independently represents an alkyl group of 1 to 5 carbon atoms, a vinyl group, or a benzyl group)
and the method comprises a step of reacting norbornadiene represented by a formula (III) shown below: 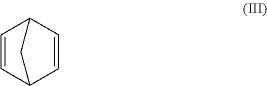
and a formic acid ester represented by a formula (IV) shown below: 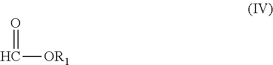
(wherein in the formula, R₁ represents an alkyl group of 1 to 5 carbon atoms, a vinyl group, or a benzyl group.)
in the presence of a ruthenium compound, a cobalt compound, a halide salt and a basic compound.

3. The method of producing a norbornanedicarboxylic acid ester according to claim 1, wherein the ruthenium compound is a ruthenium complex compound having a carbonyl ligand and a halogen ligand.

4. The method of producing a norbornanedicarboxylic acid ester according to claim 1, wherein the halide salt is a quaternary ammonium salt.

5. The method of producing a norbornanedicarboxylic acid ester according to claim 1, wherein the basic compound is a tertiary amine compound.

6. The method of producing a norbornanedicarboxylic acid ester according to claim 1, wherein the reacting is performed in the presence of a phenol compound.

7. The method of producing a norbornanedicarboxylic acid ester according to claim 1, wherein the reacting is performed in the presence of an organohalogen compound.

8. A method of producing an exo-norbornanedicarboxylic acid ester, comprising a step of separating the norbornanedicarboxylic acid ester obtained using the method of producing a norbornanedicarboxylic acid ester according to claim 1 into an endo-norbornanedicarboxylic acid ester and an exo-norbornanedicarboxylic acid ester.

9. The method of producing a norbornanedicarboxylic acid ester according to claim 6, wherein the reacting is performed in the presence of an organohalogen compound.

10. A method of producing an exo-norbornanedicarboxylic acid ester, comprising a step of separating the norbornanedicarboxylic acid ester obtained using the method of producing a norbornanedicarboxylic acid ester according to claim 9 into an endo-norbornanedicarboxylic acid ester and an exo-norbornanedicarboxylic acid ester.

11. A method of producing an exo-norbornanedicarboxylic acid ester, comprising a step of separating the norbornanedicarboxylic acid ester obtained using the method of producing a norbornanedicarboxylic acid ester according to claim 7 into an endo-norbornanedicarboxylic acid ester and an exo-norbornanedicarboxylic acid ester.

12. A method of producing an exo-norbornanedicarboxylic acid ester, comprising a step of separating the norbornanedicarboxylic acid ester obtained using the method of producing a norbornanedicarboxylic acid ester according to claim 6 into an endo-norbornanedicarboxylic acid ester and an exo-norbornanedicarboxylic acid ester.
